(19) United States
(12) Patent Application Publication
(10) Pub. No.: US 2022/0356525 A1
(43) Pub. Date: Nov. 10, 2022

(54) METHODS OF DETERMINING WHETHER PATIENTS SUFFERING FROM ACUTE MYELOID LEUKEMIA WILL ACHIEVE A RESPONSE TO AN MYC-TARGETING THERAPY

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); ASSISTANCE PUBLIQUE-HÔPITAUX DE PARIS (APHP), Paris (FR); UNIVERSITÉ DE PARIS, Paris (FR)

(72) Inventors: Alexandre PUISSANT, PARIS (FR); Raphaël ITZYKSON, PARIS (FR)

(21) Appl. No.: 17/764,642

(22) PCT Filed: Oct. 15, 2020
(86) PCT No.: PCT/EP2020/079122

§ 371 Date: Mar. 29, 2022

(30) Foreign Application Priority Data
Oct. 16, 2019 (EP) 19306350.0

(51) Int. Cl. C12Q 1/6883; A61K 31/517; A61K 31/551; A61K 31/55; A61K 31/5517; A61K 31/5377; A61P 35/02

(52) U.S. Cl. CPC C12Q 1/6883; A61K 31/517; A61K 31/551; A61K 31/55; A61K 31/5517; A61K 31/5377; A61P 35/02; C12Q 1/6869

(57) ABSTRACT

Deciphering the impact of metabolic intervention on response to anticancer therapy represents a path toward improved clinical responses. Here, the inventors identify amino acid-related pathways connected to the folate cycle whose activation predicts sensitivity to MYC-targeting therapies in acute myeloid leukemia (AML). They establish that folate restriction and deficiency of the rate-limiting folate-cycle enzyme, MTHFR—which exhibits reduced-function polymorphisms in about 10% of Caucasians—enhance resistance to MYC targeting by BET and CDK7 inhibitors in cell lines, primary patient samples and syngeneic mouse models of AML. Further, this effect is abrogated by supplementation with the MTHFR enzymatic product, CH3-THF. Mechanistically, folate cycle disturbance reduces H3K27/K9 histone methylation, and activates a SPI1 transcriptional program counteracting the effect of BET inhibition. Thus the data provide a rationale for screening MTHFR polymorphisms and the folate cycle status to exclude patients least likely and nominate those most likely to benefit from MYC-targeting therapies.

### FIELD OF THE INVENTION

The present invention is in the field of medicine, in particular oncology.

### BACKGROUND OF THE INVENTION

The rewiring of cellular metabolic activities represents a major determinant of cancer progression and is considered a hallmark of cancer (1). Metabolic reprogramming supports the acquisition and maintenance of malignant properties. The integration of high-throughput omics technologies such as metabolomics and loss-of-function screening has revolutionized our understanding of how metabolic dependencies support cancer cell proliferation (2-4). Although non-proliferating cell activity depends primarily on catabolic demands, proliferating cells must balance the divergent catabolic and anabolic requirements of sustaining cellular homeostasis while duplicating mass, and thus become dependent on a plethora of metabolic pathways that are not typically essential for normal tissue maintenance (5). From a clinical standpoint, this peculiar metabolic rewiring of neoplastic cells thereby engenders metabolic liabilities that can be exploited to design innovative therapeutic strategies, including those to increase the therapeutic index of existing anticancer therapies. For instance, it was recently observed that dietary supplementation of histidine enhances leukemic cell sensitivity to the widely used chemotherapeutic methotrexate, an antimetabolite which inhibits de novo nucleotide synthesis (6). This suggests that dietary supplementation can be leveraged to curtail the toxicity of anticancer therapies while maximizing their on-target activity.

In addition to diet-mediated enhancement of antimetabolite-based chemotherapy, metabolic perturbation may substantially influence cell response to therapies targeting major oncogenes involved in active hijacking of neoplastic cell metabolism. In that regard, MYC represents a paradigmatic oncogene as this transcription factor is deregulated in more than 50% of human cancers and reprograms many aspects of cell metabolism including glucose uptake and glycolysis, glutaminolysis, serine/glycine metabolism, and lipid biosynthesis (7). A general feature of MYC deregulation is its transcriptional regulation by super-enhancer genomic regions. These clusters of enhancers are densely occupied by transcription factors and chromatin regulators—including BET bromodomain proteins, and CDK7 and CDK9 kinases—and have been exploited over the past decade as essential targets owing to their MYC transcriptional regulator function. Therefore, indirect targeting of MYC transcription through inhibition of these essential regulators has shown great promise in pre-clinical studies, notably in AMLs harboring MLL gene fusions (8). Inhibitors targeting these chromatin remodelers are currently in phase 1-2 clinical trials in advanced solid tumors and hematologic malignancies (8-10).

The diverse metabolic alterations induced by MYC may constitute another source of unique metabolic rewiring that could be exploited to nominate new treatment strategies. These approaches may enhance cell susceptibility to its inhibition by this new class of inhibitors or enable identification of patient populations most likely to benefit from MYC-targeting therapies.

### SUMMARY OF THE INVENTION

As defined by the claims, the present invention relates to methods of determining whether patients suffering from acute myeloid leukemia will achieve a response to an MYC-targeting therapy.

### DETAILED DESCRIPTION OF THE INVENTION

Deciphering the impact of metabolic intervention on response to anticancer therapy represents a path toward improved clinical responses. Here, the inventors identify amino acid-related pathways connected to the folate cycle whose activation predicts sensitivity to MYC-targeting therapies in acute myeloid leukemia (AML). They establish that folate restriction and deficiency of the rate-limiting folate-cycle enzyme, MTHFR—which exhibits reduced-function polymorphisms in about 10% of Caucasians—enhance resistance to MYC targeting by BET and CDK7 inhibitors in cell lines, primary patient samples and syngeneic mouse models of AML. Further, this effect is abrogated by supplementation with the MTHFR enzymatic product, CH3-THF. Mechanistically, folate cycle disturbance reduces H3K27/K9 histone methylation, and activates a SPI1 transcriptional program counteracting the effect of BET inhibition. Thus the data provide a rationale for screening MTHFR polymorphisms and the folate cycle status to exclude patients least likely and nominate those most likely to benefit from MYC-targeting therapies.

Accordingly, the first object of the present invention relates to a method of determining whether a patient suffering from acute myeloid leukemia will achieve a response to an MYC-targeting therapy comprising determining in a nucleic acid sample obtained from the subject the presence or absence of at least one genetic variant in the MTHFR gene wherein the presence of said genetic variant indicates that the patient will not achieve a response to the MYC-targeting therapy whereas the absence of said genetic variant indicates that the patient will achieve a response to the MYC targeting therapy.

As used herein, the term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

MYC-targeting therapies are well known in the art. Indeed a vast array of strategies, both direct and indirect, have been employed for targeting MYC by exploiting its multiple regulatory mechanisms, including MYC transcription and mRNA stability, MYC protein stability and degradation, as well as MYC binding to its interactome. Examples include inhibitors of MYC transcription with direct G-quadruplex stabilizers, antisense oligonucleotides that induce MYC mRNA degradation, aberrant splicing of MYC pre-mRNA or translation block, as well as short-interfering RNAs. Indirect MYC suppression may also achieve via inhibitors of regulators of MYC protein stability and turnover (e.g., GSK3, Ras/Raf/MAPK, PP2A, FBW7, SKP2, hTERT), inhibitors of pathways that are involved in MYC translation (e.g., MAPK, mTORC1 and FOXO3a), and inhibitors of MYC chromatin remodeling and transcription. In particular, bromodomain inhibitors, CDK7 inhibitors, and CDK9 inhibitors are particularly suitable for inhibiting MYC expression at the transcriptional level. Thus in some embodiments, the MYC-targeting therapy according to the present invention consists in administering the patient with a bromodomain inhibitor, a CDK7 inhibitor, or a combination thereof.

In some embodiments, the bromodomain inhibitor is a BET inhibitor. As used herein the term “BET inhibitor” has its general meaning in the art and refers to any molecule or compound that can prevent or inhibit the binding of the bromodomain of at least one BET family member to acetyl-lysine residues of proteins. It is to be understood that a BET inhibitor may inhibit only one BET family member or it may inhibit more than one or all BET family members. In some embodiments, the BET inhibitor is a small molecule compound that binds to the binding pocket of the first bromodomain of a BET family member (e.g., BRD1, BRD2, BRD3, BRD4, BRD7, and BRDT). The BET inhibitor may be any molecule or compound that inhibits a BET, including nucleic acids such as DNA and RNA aptamers, antisense oligonucleotides, siRNA and shRNA, small peptides, antibodies or antibody fragments, and small molecules such as small chemical compounds. Examples of BET inhibitors are described in JP2009028043, JP2009183291, WO2011054843, WO2011054848, WO2009084693, WO2009084693, WO 2011054844, WO 2011054846, WO2011054851, WO2011143669, and WO2011143660, US2012028912, Filippakopoulos et al. Bioorg Med Chem. 20(6): 1878-1886, 2012; Chung et al. J Med Chem. 54(11):3827-38, 2011; and Chung et al. J Biomol Screen. 16(10):1170-85, 2011, which are incorporated herein by reference. Examples of BET inhibitors known in the art include, but are not limited to, RVX-208 (Resverlogix), PFI-1 (Structural Genomics Consortium), OTX015 (Mitsubishi Tanabe Pharma Corporation), BzT-7, and GSK525762A (iBET, GlaxoSmithKline). In some embodiments, the BET inhibitor is JQ1 that is also known as tert-butyl 2-[(9S)-7-(4-chlorophenyl)-4,5,13-trimethyl-3-thia-1,8,11,12-tetrazatricyclo[8.3.0.02,6]trideca-2(6),4,7,10,12-pentaen-9-yl]acetate and is disclosed in WO2009084693. In some embodiments the BET inhibitor is I-BET-762 (also known as: GSK-525762A). In some embodiments, the BET inhibitor is LY294002 (Dittmann et al., “The Commonly Used PI3-Kinase Probe LY294002 is an Inhibitor of BET Bromodomains”. ACS Chemical Biology: 2013, 131210150813004).

As used herein, the term “selective CDK7 inhibitor” refers to a CDK7 inhibitor that reduces the activity of CDK7 more than it reduces the activity of any other cyclin-dependent kinase (“CDK”). For the purposes of this application, the CDK inhibitors flavopiridol, BMS-387032, PHA-793887, Roscovitine are not selective CDK7 inhibitors as each has been shown to have a lower inhibitory activity toward CDK7 than toward at least one other CDK (see Table 1, Kwiatkowski et al. (2014); Nature 51 1 (751 1)).

In some embodiments, the MYC targeting therapy MYC-targeting therapy according to the present invention consists in administering the patient with an inhibitor of expression. An “inhibitor of expression” refers to a natural or synthetic compound that has a biological effect to inhibit the expression of a gene such as BRD1, BRD2, BRD3, BRD4, BRD7, or BRDT, as well as CD7 or CDK9. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of the targeted mRNA by binding thereto and thus preventing protein translation or increasing mRNA degradation, thus decreasing the level of the targeted, and thus activity, in a cell. For example, antisense oligonucleotides of at least about 15 bases and complementary to unique regions of the mRNA transcript sequence encoding the targeted can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. Gene expression can be reduced by contacting a patient or cell with a small double stranded RNA (dsRNA), or a vector or construct causing the production of a small double stranded RNA, such that gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing the target of interest. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. In a particular embodiment, the endonuclease is CRISPR-cas. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

As used herein, the term “nucleic acid sample” refers to any biological sample isolated from the subject liable to contain nucleic acid for the purpose of the present invention. Samples can include by way of example and not limitation, body fluids (e;g. saliva) and/or tissue extracts such as homogenates or solubilized tissue obtained from the subject. In some embodiments, the sample is a blood sample. The term “blood sample” means any blood sample derived from the patient that contains nucleic acids. Peripheral blood is preferred, and mononuclear cells (PBMCs) are the preferred cells. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art. The template nucleic acid need not be purified. Nucleic acids may be extracted from a sample by routine techniques such as those described in Diagnostic Molecular Microbiology: Principles and Applications (Persing et al. (eds), 1993, American Society for Microbiology, Washington D.C.).

As used herein, the term “MTHFR” refers to the gene encoding for the MTHFR. The MTHFR gene is known per and is available under the reference ENSG00000177000 in the Ensembl Gene Database.

As used herein, the term “genetic variant” has its general meaning in the art and denotes any of two or more alternative forms of a gene occupying the same chromosomal locus. The alteration typically consists in a substitution, an insertion, and/or a deletion, at one or more (e.g., several) positions in the gene. Genetic variation arises naturally through mutation, and may result in phenotypic polymorphism within populations. Gene mutations can be silent (no change in the encoded polypeptide) or may encode polypeptides having altered amino acid sequence. The term is also known as “polymorphism”.

In some embodiments, the genetic variant is located in the promoter. In some embodiments, the genetic variant is located in an intron. In some embodiments, the genetic variant is located in an exon.

In some embodiments, the genetic variant is present is heterozygous (i.e. present in only one allele) or homozygous (i.e. present in the 2 alleles).

In some embodiments, the presence or absence of the c.677C>T or c.1298A>C is determined, wherein the presence of 677 CC or 1298 AA genotype indicate that the patient will not achieve a response to the MYC-targeting therapy.

Detecting the genetic variant may be determined according to any genotyping method known in the art. Typically, common genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, sequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection. Various methods for detecting polymorphisms include, but are not limited to, methods in which protection from cleavage agents is used to detect mismatched bases in RNA/RNA or RNA/DNA, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis. Sequence variations at specific locations can also be assessed by nuclease protection assays such as RNase and SI protection or chemical cleavage methods.

In some embodiments, genotyping is performed using the TaqMan assay, which is also known as the 5′ nuclease assay. The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced. During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present. Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the nucleic acids of the present invention are useful in diagnostic assays for stenosis and related pathologies, and can be readily incorporated into a kit format.

Another method for genotyping the nucleic acids of the present invention is the use of two oligonucleotide probes in an Oligonucleotide Ligation Assay (OLA). In this method, one probe hybridizes to a segment of a target nucleic acid with its 3′ most end aligned with the nucleic acid site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the nucleic acid site. If there is a mismatch, efficient ligation cannot occur. After the reaction, the ligated probes are separated from the target nucleic acid molecule, and detected as indicators of the presence of a nucleic acid sequence. OLA may also be used for performing nucleic acid detection using universal arrays, wherein a zipcode sequence can be introduced into one of the hybridization probes, and the resulting product, or amplified product, hybridized to a universal zip code array. Alternatively OLA may be used where zipcodes are incorporated into OLA probes, and amplified PCR products are determined by electrophoretic or universal zipcode array readout. Alternatively one may use SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In some other embodiments, PCR (or another method of nucleic acid amplification) is carried out prior to OLA.

Another method for genotyping is based on mass spectrometry. Mass spectrometry takes advantage of the unique mass of each of the four nucleotides of DNA. Nucleic acids can be unambiguously genotyped by mass spectrometry by measuring the differences in the mass of nucleic acids having alternative nucleic acid alleles. MALDI-TOF (Matrix Assisted Laser Desorption Ionization—Time of Flight) mass spectrometry technology is preferred for extremely precise determinations of molecular mass, such as for SNPs. Numerous approaches to genotype analysis have been developed based on mass spectrometry. Preferred mass spectrometry-based methods of nucleic acid genotyping include primer extension assays, which can also be utilized in combination with other approaches, such as traditional gel-based formats and microarrays. Typically, the primer extension assay involves designing and annealing a primer to a template PCR amplicon upstream (5′) from a target nucleic acid position. A mix of dideoxynucleotide triphosphates (ddNTPs) and/or deoxynucleotide triphosphates (dNTPs) are added to a reaction mixture containing template. For example, in some embodiments this is a SNP-containing nucleic acid molecule which has typically been amplified, such as by PCR. Primer and DNA polymerase may further be added. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the nucleic acid position. If the primer is several nucleotides removed from the target nucleic acid position, the only limitation is that the template sequence between the 3′ end of the primer and the nucleic acid position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer. The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position.

Detecting the genetic variant may also be performed by sequencing. A variety of automated sequencing procedures can be used, including sequencing by mass spectrometry. The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing. Nucleic acid sequences can also be determined by employing a high throughput mutation screening system, such as the SpectruMedix system.

Other methods that can be used to genotype the nucleic acids of the present invention include single-strand conformational polymorphism (SSCP), and denaturing gradient gel electrophoresis (DGGE). SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at nucleic acid positions. DGGE differentiates nucleic acid alleles based on the different sequence-dependent stabilities and melting properties inherent in polymorphic DNA and the corresponding differences in electrophoretic migration patterns in a denaturing gradient gel.

Sequence-specific ribozymes can also be used to score nucleic acids, in particular SNPs, based on the development or loss of a ribozyme cleavage site. Perfectly matched sequences can be distinguished from mismatched sequences by nuclease cleavage digestion assays or by differences in melting temperature. Thus, for example, if the SNP affects a restriction enzyme cleavage site, the SNP can be identified by alterations in restriction enzyme digestion patterns, and the corresponding changes in nucleic acid fragment lengths determined by gel electrophoresis. Genotyping can include the steps of, for example, collecting the sample, isolating nucleic acids (e.g., genomic DNA, mRNA or both) from the cells of the sample, contacting the nucleic acids with one or more primers which specifically hybridize to a region of the isolated nucleic acid containing a target nucleic acid region of interest under conditions such that hybridization and amplification of the target nucleic acid region occurs, and determining the nucleotide present at the nucleic acid position of interest, or, in some assays, detecting the presence or absence of an amplification product (assays can be designed so that hybridization and/or amplification will only occur if a particular nucleic acid sequence allele is present or absent). In some assays, the size of the amplification product is detected and compared to the length of a control sample; for example, deletions and insertions can be detected by a change in size of the amplified product compared to a normal genotype. Methods of comparing the identity of two or more sequences may be performed by any reasonable means, including programs available in the Wisconsin Sequence Analysis Package version 9.1 (Genetics Computer Group, Madison, Wis., USA). Other programs such as BESTFIT may be used to find the “local homology” algorithm of Smith and Waterman and finds the best single region of similarity between two sequences. Further, programs such as GAP may be used, which aligns two sequences finding a “maximum similarity.” Preferably, % identities and similarities are determined when the two sequences being compared are optimally aligned. Other programs for determining identity and/or similarity between sequences are also known in the art, for instance the BLAST family of programs, available from the National Center for Biotechnology Information (NCB), Bethesda, Md., USA) and FASTA, available as part of the Wisconsin Sequence Analysis Package.

Methods of detecting the genetic variants according to the present invention are well known in the art and typically include the method described in Lajin B, Alachkar A, Sakur A A (February 2012). “Triplex tetra-primer ARMS-PCR method for the simultaneous detection of MTHFR c.677C>T and c.1298A>C, and MTRR c.66A>G polymorphisms of the folate-homocysteine metabolic pathway”. Molecular and Cellular Probes. 26 (1): 16-20. doi:10.1016/j.mcp.2011.10.005. PMID 22074746.

The method is thus particularly suitable for discriminating responder from non-responder. As used herein the term “responder” in the context of the present disclosure refers to a patient that will achieve a response, i.e. a patient where the cancer is eradicated, reduced or improved. According to the invention, the responders have an objective response and therefore the term does not encompass patients having a stabilized cancer such that the disease is not progressing after the MYC-targeting therapy. A non-responder or refractory patient includes patients for whom the cancer does not show reduction or improvement after the MYC-targeting therapy. According to the invention the term “non-responder” also includes patients having a stabilized cancer. Typically, the characterization of the patient as a responder or non-responder can be performed by reference to a standard or a training set. The standard may be the profile of a patient who is known to be a responder or non-responder or alternatively may be a numerical value. Such predetermined standards may be provided in any suitable form, such as a printed list or diagram, computer software program, or other media. When it is concluded that the patient is a non-responder, the physician could take the decision to stop the MYC-targeting therapy to avoid any further adverse sides effects and to administer the patient with an additional therapy. In particular, when it is concluded that the patient will not achieve a response to the MYC-targeting therapy, then the patient is administered with the MYC-targeting therapy in combination with a therapeutically effective amount of 5-methyltetrahydrofolate (5-CH3-THF).

Thus a further object relates to a method of treating acute myeloid leukemia in a patient in need thereof comprising the steps consisting of i) determining in a nucleic acid sample obtained from the subject the presence or absence of at least one genetic variant in the MTHFR gene and ii) administering the patient with a MYC-targeting therapy when the absence of the genetic is detected or administering the patient with a MYC-targeting therapy in combination with a therapeutically effective amount of 5-methyltetrahydrofolate when the presence of the genetic variant is detected.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is a MYC-targeting drug and wherein the other drug is 5-methyltetrahydrofolate. In some instance, the combination of the present invention results in the synthetic lethality of the cancer cells.

In some embodiments, the administration of 5-methyltetrahydrofolate is performed by dietary supplementation.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

The invention will be further illustrated by the following figures and examples. However, these examples and figures should not be interpreted in any way as limiting the scope of the present invention.

### FIGURES

FIG. 1: C677T and A1298C MTHFR variants promote resistance to BET Inhibitors.

(A) Allelic discrimination plots depicting polymorphic status of MTHFR at C677 and A1298. Five CRISPR/Cas9-edited KG1a clones with various MTHFR genetic variants on C677 and A1298 were selected.

(B) Distribution of IC₅₀ with OTX015 for 6 days of CRISPR/Cas9-edited KG1a clones exhibiting various MTHFR genetic variants (n=5 clones per genotype). *p-value by nonparametric Mann-Whitney test. Error bars represent mean±SD. Experiment performed at least two independent times.

(C) Colony formation from CRISPR/Cas9-edited KG1a clones exhibiting various MTHFR genetic variants with 1 μM OTX015. Results represent average of triplicate assays. *p-value≤0.05 by Welch's t-test versus MTHFR 677CC & A1298AA clone with OTX015. Error bars represent mean±SD.

(D) Distribution of IC₉₀ with OTX015 for 5 days of 16 MLL-translocated patient samples with AML divided into 2 subgroups according to MTHFR genetic status at C677, A1298. p-value by nonparametric Mann-Whitney test. Error bars represent mean±SD.

(E) MLL-AF9-transformed GFP-positive granulomonocytic progenitors (Sca-1⁻/c-KIT⁺/CD16/32⁺/CD34⁺/MPO⁺) from homozygous wild-type (+/−), or heterozygous (+/−) and homozygous (−/−) Mthfr knockout mice transplanted into sublethally-irradiated recipient mice before treatment with vehicle or 50 mg/kg JQ1 for 7 days. Proportion of GFP-positive MLL-AF9 leukemic cells in bone marrow from five mice per group. p-value by Mann-Whitney test. n.s, nonsignificant (p>0.05). Error bars represent mean±SD.

(F-G) Fold-change in OTX015 sensitivity for 5 days in AML cell lines (F) or CRISPR/Cas9-edited KG1a clones (G) expressing a construct encoding either empty control or DD-tagged wild-type MTHFR divided into two groups according to MTHFR genotypes at C677, A1298. Results shown as fold change of IC₅₀ normalized to average empty vector condition. *p-value by nonparametric Mann-Whitney test. n.s, nonsignificant (p>0.05). Error bars represent mean±SD.

(H-I) Fold-change in OTX015 sensitivity for 5 days of IMS-M2 and U937 cells infected with control or two MTHFR-directed shRNAs (H) or indicated CRISPR/Cas9-edited KG1a clones (I) with 50 μM 5-CH3 THF. Results shown as fold change of IC₅₀ normalized to average untreated condition. *p-value by nonparametric Mann-Whitney test. n.s, nonsignificant (p>0.05). Error bars represent mean±SD.

Each experiment in G-J performed at least two independent times.

(J) Distribution of IC₉₀ to OTX015 for 5 days and 50 μM 5-CH3 THF treatment of three MLL-translocated AML patient samples. Square represents a patient with MTHFR 677 CT & 1298 AC genotype; the two others exhibit MTHFR 677 CC & A1298 CC genotype. p-value by nonparametric Mann-Whitney test. Error bars represent mean±SD.

### EXAMPLE

Methods

Metabolomic Analyses

To determine relative levels of intracellular metabolites, extracts were prepared and analyzed by LC/MS/MS. 16 hours before extraction, 15×10⁶ U937 cells were plated in quadruplicate in folic acid-free RPMI 1640 supplemented with 10% dialyzed FBS and 100 units/ml penicillin/streptomycin+/−1 mg/L folic acid. Metabolites were extracted on dry ice with 4 mL of 80% methanol (−80° C.), as described previously (38). Metabolite production compared to average control condition is represented by a color gradient from blue to red. Additional information is provided in the supplementary material and methods section.

Pooled CRISPR/Cas9 Epigenetic Screen

OCI-AML2 cells were seeded into six-well plates at a density of 3×10⁶ cells per well and transduced at MOI 0.2. 144×10⁶ cells were transduced in 48 wells total. 24 hours post-transduction, cells were replated into puromycin-containing media. A sample was collected at 48 hours post-puromycin to confirm library coverage in the transduced population. Transduced cells were expanded in puromycin for 10 days before drug, when the transduced population was split into vehicle (DMSO) and JQ1 conditions and maintained for two weeks. Deep sequencing was performed by Hudson Alpha Institute for Biotechnology on an Illumina Nextseq platform (75 bp, single-ended) to identify differences in library composition. Additional information is provided in the supplementary material and methods section.

CRISPR/Cas9-Mediated Introduction of Single Nucleotide Polymorphisms On MTHFR

Top and bottom sgRNAs targeting the C677 and A1298 MTHFR sites were annealed and phosphorylated as previously described (24157548), and ligated into the BbSSI-digested pSpCas9(BB)-2A-GFP (PX458) vector (Addgene, #48138) to generate PX458_sgC677 and PX458_sgA1298 constructs. 1×10⁶ KG-1a cells were washed in PBS before resuspension in 100 μl Opti-MEM medium (ThermoFisher Scientific, #31985-047). 10 μg PX458_sgC677 or PX458_sgA1298 vector and 0.3 μM SSODN_C677 or SSODN_A1298 were added into the mix of Opti-MEM and cells to generate isogenic KG-1a clones expressing either MTHFR 677 C>T or A1298 A>C genetic variant, respectively. Additional information is provided in the supplementary material and methods section.

Integrated ChIP- and RNA-Sequencing Analysis.

SICER-identified significantly decreased H3K9me2 (14651 regions in IMSM2; 4842 regions in U937) and H3K27me3 (4048 regions in IMSM2; 4855 regions in U937) peaks in OTX−FA versus OTX+FA conditions were annotated for the 2 nearest genes using GREAT (39) within a 50 kb window upstream and downstream. The union of lists of annotated genes per cell line was kept defining a list of all genes in the vicinity of lost H3K9me2 or H3K27me3 (3961 genes for IMSM2; 4416 genes for U937).

Significantly upregulated genes (Fold Change>1.2; p-value<0.05) identified by RNAseq experiments comparing OTX−FA versus OTX+FA conditions were selected (623 genes in IMSM2; 1342 genes in U937) compared to the lists of genes proximal to decreased H3K9me2 and H3K27me3 regions identified previously leading to an overlap of 93 genes in IMSM2 and 202 genes in U937. Additional information is provided in the supplementary material and methods section.

In Silico Tests for Pearson Correlation Calculation Between MYC-Related and Metabolic Gene Sets

Single-sample GSEA (ssGSEA) was used to calculate separate enrichment scores for each pairing of a sample whose transcriptomic data was available from TCGA-LAML (40) or GSE14468 {Wouters, 2009 #42 and a given gene set (queried from MSigDB for MYC and KEGG-related gene signatures or manually curated from BIOCYC for other metabolic gene sets). A Pearson correlation matrix was computed between each ssGSEA score for the core MYC signature and all gene sets of interest obtained across all patients from a given cohort. Connected metabolic pathways were clustered based on the median of Pearson correlation scores obtained from each individual pathway.

In Vivo Transplantation

The French National Committee on Animal Care reviewed and approved all mouse experiments. Sample size was influenced by historical penetrance and consistency of MLL-AF9-driven in vivo models. For the Mthfr knockout AML mouse model, BALB/cJ mice were purchased from Charles River Laboratories. Each recipient mouse was transplanted with transduced Scal⁻/c-Kit⁺ myeloid progenitors sorted from the total bone marrow of three donor mice. Approximatively two months post-transplantation, sick mice were euthanized and bone marrow harvested before flow-based sorting of MLL-AF9-positive granulo-monocytic bone marrow progenitor population (GFP⁺/Sca-1⁻/c-Kit⁺/Cd16/32⁺/Cd34⁺) and PCR-based confirmation of Mthfr knockout status. 0.2×10⁶ MLL-AF9-positive Mthfr^{+/+}, Mthfr^{+/-}, and Mthfr^{-/-} cells were then reinjected into sublethally-irradiated secondary recipient mice treated daily by intraperitoneal injection with 50 mg/kg JQ1 (10% DMSO+90% G5W).

For folate starvation experiments, 5-week old male C57BL/6J mice were given regular or folate-deficient casein-reconstituted diet (Safe-Diets) for 4 weeks before sublethal irradiation (350cGy) and injection with 0.2×10⁶ MLL-AF9-positive L-GMP cells before treatment with 35 mg/kg or 50 mg/kg JQ1 (10% DMSO+90% G5W) for 7 days. For Mthfr knockdown experiments, MLL-AF9-positive L-GMP cells were infected with a control or two Mthfr-directed shRNAs cloned into an MSCV-miRE-SV40-eBFP vector modified from the MSCV-miRE_shBRD9_561-SV40-GFP vector (Addgene, #75139) by substitution of the GFP cassette with an eBFP fluorescent marker. 0.2×10⁶ infected cells were injected into sublethally-irradiated 5-week old C57BL/6J recipient mice. Additional information is provided in the supplementary material and methods section.

Results

Folate Restriction Promotes Resistance to Therapies Targeting MYC Expression.

BET proteins interact with acetylated histones in active regulatory domains (promoters and enhancers) and promote RNA PolII activity. Despite the general nature of this mechanism, BET inhibitors have shown selective effects on gene expression through suppression of YC and MYC-related transcriptional programs (8, 11, 12). Two cohorts of AML patients (TCGA-LAML, n=198 and GSE14468, n=526) were queried with multiple gene sets related to MYC transcriptional programs as well as vitamin, amino acid, and nucleotide metabolism using single sample Gene Set Enrichment Analysis (ssGSEA). Correlation between gene sets was queried across all patients from each cohort to pinpoint potential crosstalk between active MYC programs and specific metabolic pathways (data not shown). Whereas many pathways related to nucleotide (pyrimidine, purine), tetrahydrofolate salvage, lysine, cysteine/methionine, arginine/proline alanine/aspartate/glutamate and valine/leucine/isoleucine metabolism were strongly correlated with active MYC signatures, those associated with folate cycle and biosynthesis, histidine, glycine/serine/threonine, beta-alanine, phenylalanine/tyrosine, and tryptophan metabolism were all poorly correlated with active MYC signatures, suggesting that these pathways may either act as negative regulators of MYC program or that they are negatively regulated by MYC (data not shown). To investigate this hypothesis, three AML cell lines (KG1a, IMS-M2, and U937) were grown in conditioned media deprived of amino acids or vitamins belonging to the metabolic pathways poorly correlated with active MYC signatures and were then treated with a BET inhibitor, OTX015 (13) (data not shown). Although phenylalanine and histidine starvation increased resistance to OTX015 in a subset of AML cell lines, folic acid starvation consistently enhanced resistance to OTX015 in all cell lines tested.

To investigate further the relationship between folic acid starvation and increased BET inhibitor resistance, we compiled a panel of AML cell lines harboring diverse genetic alterations. According to our previous observation, folic acid-starved media significantly increased the area under curve (AUC) and half-maximal inhibitory concentration (IC₅₀) of OTX015 versus standard media (data not shown). The effect of folic acid removal was also evaluated in cell lines treated either with a second BET inhibitor, JQ1, or with THZ-1, a covalent inhibitor of CDK7, another key factor in transcriptional elongation at super-enhancer regions and a strong inhibitor of MYC expression (data not shown). In both conditions, folic acid withdrawal significantly dampened AML cell line sensitivity to JQ1 and THZ-1. Previous studies have focused on the role of a specific BET family member, BRD4, which is potently inhibited by JQ1 and OTX015 and serves as one of the main targets triggering the deleterious effect of these compounds. Therefore, we studied selective suppression of BRD4 by short hairpin RNA (shRNA) in two AML cell lines. As seen with JQ1 and OTX015 treatments, there was a striking viability decrease upon BRD4 knockdown, which was significantly attenuated in folic acid-starved condition (data not shown). To further characterize the phenotypic consequences of folic acid withdrawal on the sensitivity of AML cells to OTX015, we performed colony formation assays and established that a 125-fold decrease in folic acid concentration (from 1 mg/L to 0.008 mg/L in standard versus conditioned media, respectively) significantly increased the number of U937 cell colonies in presence of OTX015 (data not shown). This was accompanied by a decrease in apoptosis in response to OTX015 (data not shown). Moreover, folic acid withdrawal markedly increased the IC₅₀ of OTX015 in a cohort of MLL-translocated primary patient samples with AML (data not shown).

To control for bias of in vitro culture conditions on the increased BET inhibitor resistance induced by folate starvation, mice injected with MLL-AF9-driven AML cells were fed with regular or no folic acid diet prior to JQ1 treatment. Mice fed folate-restricted diet exhibited increased homocysteine levels (hyperhomocysteinemia) in plasma versus those fed with regular diet, confirming that folate starvation recapitulated physiological features reported in previous studies (14, 15) (data not shown). Consistent with our in vitro results, the anti-leukemic activity of JQ1 was markedly attenuated in animals fed no folate diet versus those fed regular diet (data not shown).

Depletion of the Folate-Cycle Rate-Limiting Enzyme, MTHFR, Enhances Resistance to BET Inhibitors.

Dietary folic acid undergoes several conversion steps into intermediate metabolites before generation of 5,10-methylene-THF (5,10-CH2 THF). 5,10-CH2 THF is then either reduced in the folate cycle or oxidized in the formate-producing tetrahydrofolate salvage cycle, depending on cellular needs. Despite the fact that folic acid can ultimately be processed through these two interconnected pathways, we observed in our previous ssGSEA analyses that active MYC gene signatures were highly correlated with activation of tetrahydrofolate salvage cycle, but not with folate cycle-related gene sets (data not shown). This implies some negative regulatory relationship between MYC and the folate cycle, prompting us to investigate further the consequences of the suppression of each enzyme from the folate cycle on the sensitivity to OTX015. Five central enzymes control the multi-step conversion of dietary folate into 5,10-methylene-THF (5,10-CH2 THF) before its reduction to 5-methyl-THF (5-CH3 THF) and recycling to THF. We infected IMS-M2 AML cells with two hairpins that markedly reduced their expression levels (data not shown). While hairpins targeting serine hydroxymethyltransferase 1 (SHMT1) and 5-methyltetrahydrofolate-homocysteine methyltransferase (MTR) did not significantly alter response to OTX015, those targeting the two dihydrofolate reductase isoenzymes (DHFR and DHFR2), as well as 5,10-methylenetetrahydroflate reductase (MTHFR), significantly increased the IC₅₀ of OTX015 by averages of 3- and 7-fold, respectively (data not shown). Given that MTHFR is the rate-limiting enzyme in the folate cycle and that its knockdown induced the most striking decrease in sensitivity to OTX015 in IMS-M2 cells, we pursued our investigation of the effect of the two MTHFR-directed shRNAs on OTX015 response by including additional AML cell lines: KG1a, OCI-AML2, and U937 (data not shown). As seen previously with folate starvation, MTHFR suppression substantially weakened the response of all tested cell lines to increasing doses of OTX015 (data not shown). This effect was associated with a significant increase in colony number of MTHFR-depleted cells versus shCT-infected cells upon OTX015 treatment (data not shown). Finally, we abrogated Mthfr expression in primary murine MLL-AF9-driven AML cells using two Mthfr-directed shRNAs before treatment of engrafted mice with JQ1 (data not shown). In animals transplanted with Mthfr-depleted blasts, leukemic burden was not significantly decreased by JQ1 treatment compared to control counterparts which exhibited striking sensitivity to JQ1 (data not shown). Thus, the overall survival of mice injected with Mthfr-depleted cells was significantly shorter than mice with control leukemic cells after JQ1 treatment (data not shown). Collectively, these data nominated MTHFR as a critical folate cycle enzyme whose impairment increases resistance to MYC-targeting therapies.

Frequent Polymorphisms in Human Populations Associated with Reduced MTHFR Activity Promote Resistance to BET Inhibitors.

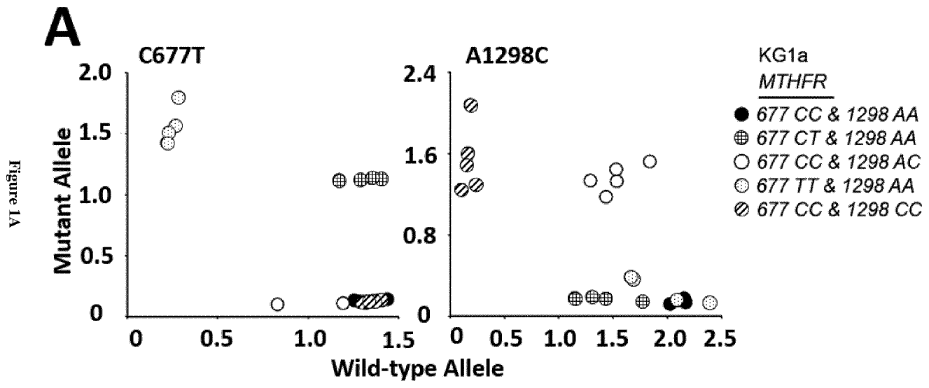

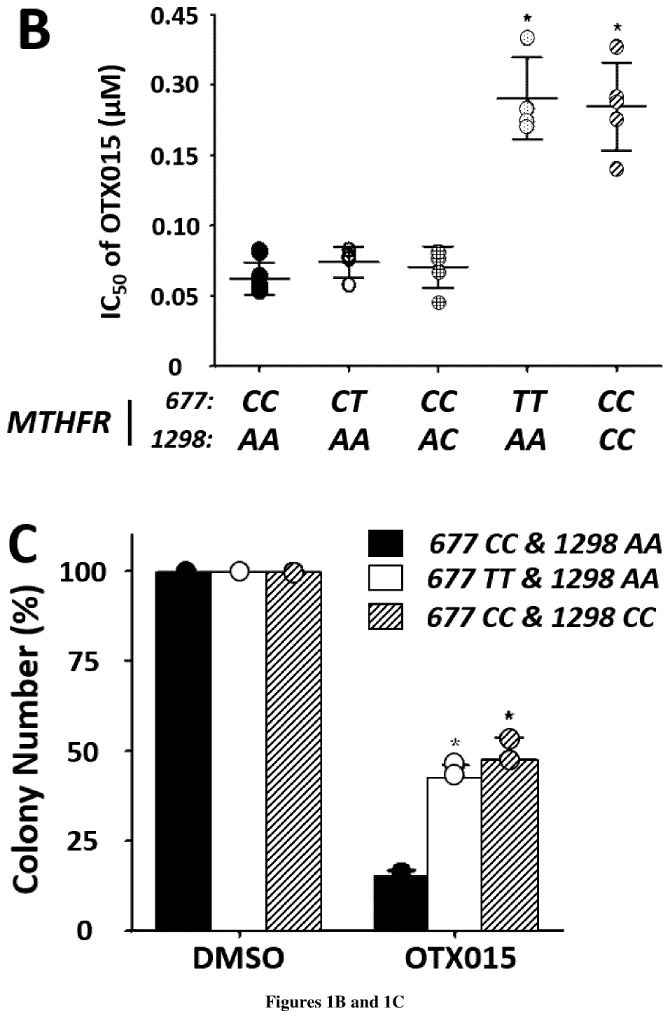

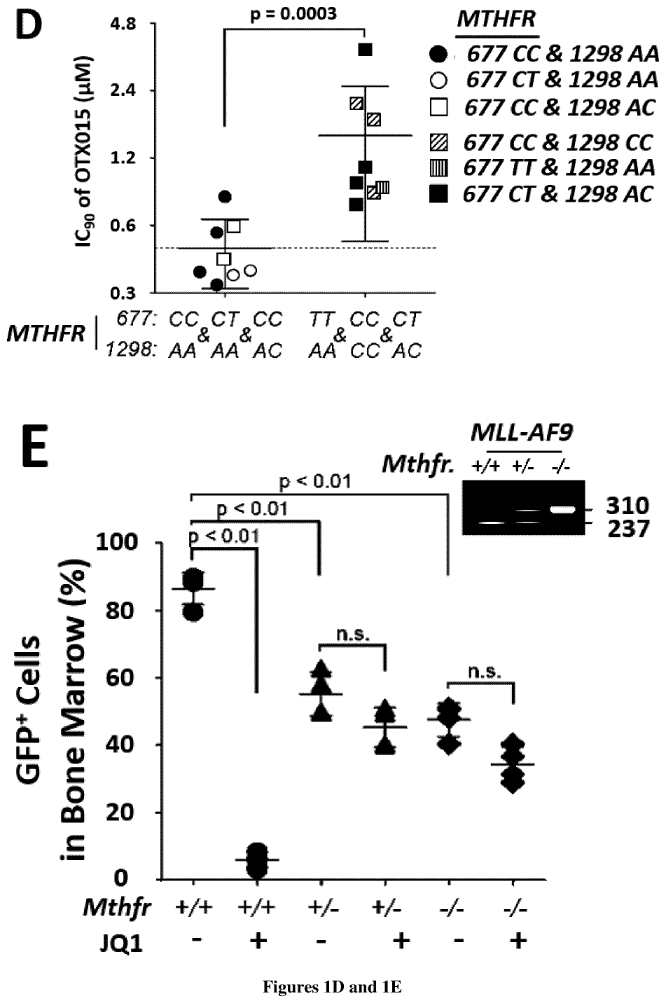

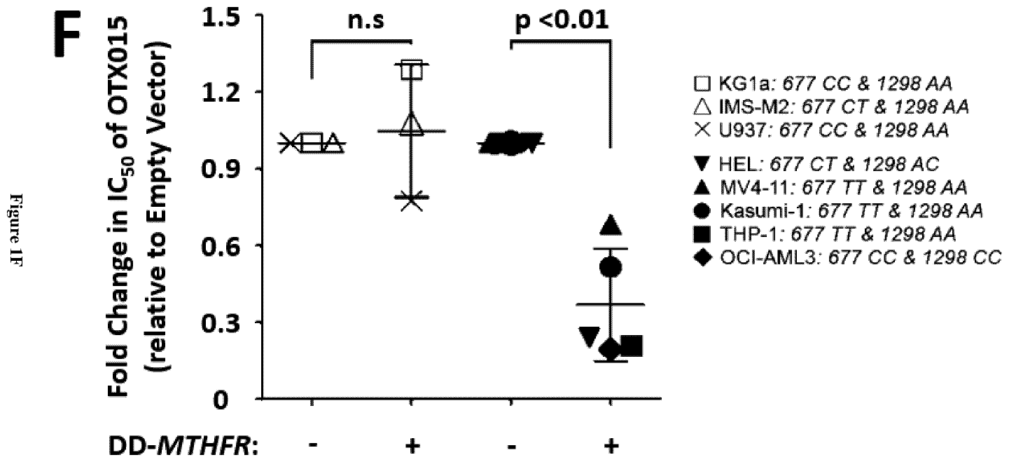

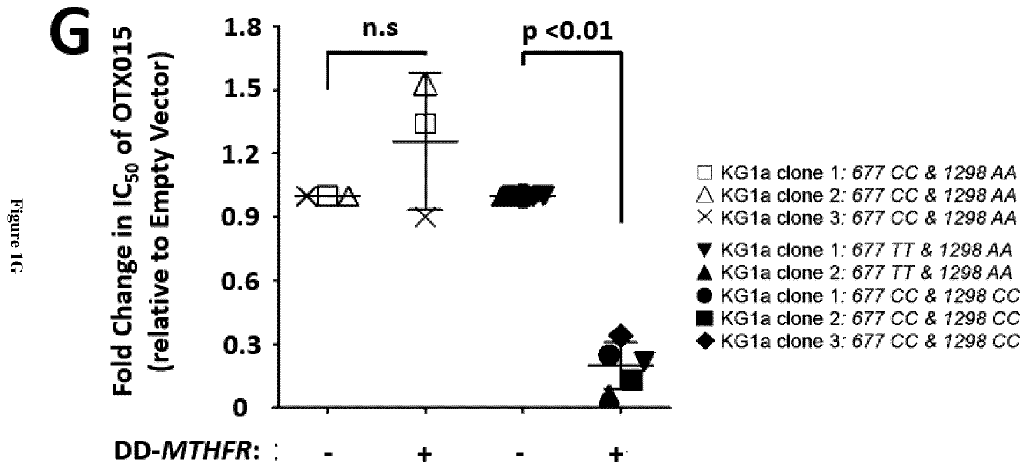

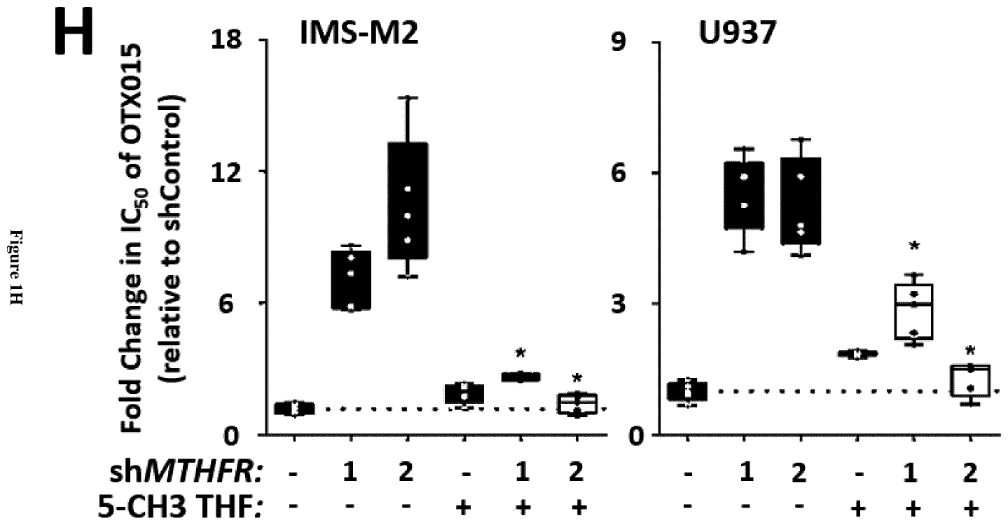

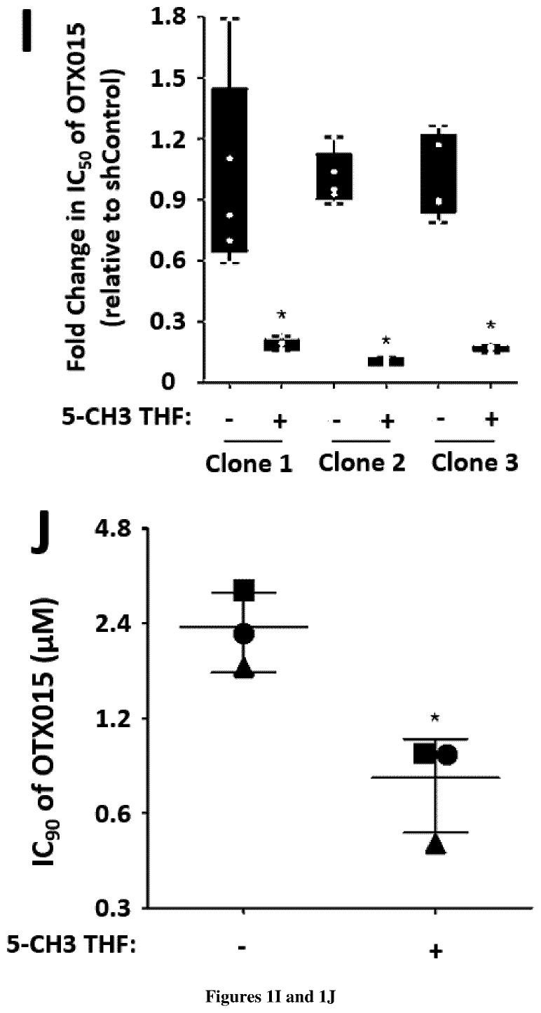

Previous studies have extensively characterized two common MTHFR genetic variants, C677T and A1298C, encoding MTHFR enzyme variants with reduced activity, resulting in hyperhomocysteinemia in human carriers. In particular, the homozygous variant 677 TT and 1298 CC genotypes are present in ˜10% of Caucasians and display only 30% and 60% of the homozygous wild-type 677 CC and 1298 AA enzyme activities, respectively (16, 17). To explore the impact of these MTHFR variants on BET inhibitor resistance, we deployed a CRISPR-Cas9-based method to edit the genome of KG1a cells and introduce these two non-synonymous single nucleotide polymorphisms in MTHFR, thereby generating isogenic cell lines exhibiting all variant combinations. Five KG1a clones either 1) wild-type 677 CC and 1298 AA, 2) heterozygous 677 CT or 1298 AC, or 3) homozygous 677 TT or 1298 CC, were selected and genotyped via allelic discrimination technique (FIG. 1A). Although the clones heterozygous for any of the two variants had similar sensitivity to OTX015 as wild-type clones, 677 TT and 1298 CC homozygous KG1a clones were significantly more resistant to OTX015 than their wild-type counterpart (FIG. 1B). Furthermore, the number of colonies from 677 TT and 1298 CC homozygous KG1a clones was substantially higher than those derived from wild-type KG1a clones upon OTX015 treatment (FIG. 1C). Based on these results, we divided a cohort of 16 MLL-translocated primary AML patient cells into two subgroups by MTHFR genotype: wild-type and single-heterozygous versus homozygous and compound heterozygous for any of two MTHFR variants. Patients displaying a homozygous and compound heterozygous MTHFR genotype for any of the two variants responded significantly less to OTX015 than those with wild-type homozygous and heterozygous MTHFR genotypes (FIG. 1D). Finally, we engineered a tractable MLL-AF9-driven mouse model of AML in which Mtfhr genotype status was either homozygous wild-type, heterozygous or homozygous knockout. We established that loss of a single copy of Mthfr which phenocopies in mice a partial impairment in MTHFR activity caused by non-synonymous single nucleotide polymorphisms on MTHFR (18), was sufficient to attenuate sensitivity to JQ1 of MLL-AF9-driven leukemias (FIG. 1E).

Because impaired MTHFR activity promotes BET inhibitor resistance, we hypothesized that overexpression of wild-type MTHFR may conversely enhance sensitivity of AML cells lacking proper MTHFR function due to the presence of the two genetic C677T and A1298C MTHFR variants. Exogenous DD-tagged wild-type MTHFR was overexpressed in AML cells exhibiting various 677 CC and 1298 AA genotypes (Data not shown). This construct markedly increased sensitivity to OTX015 of AML cell lines displaying homozygous and compound heterozygous MTHFR genotypes for any of the two variants in comparison with cell lines exhibiting either a wild-type or heterozygous 677 CT genotype (FIG. 1F). A similar analysis performed on the isogenic MTHFR-edited KG1a clones showed that those displaying homozygous 677 TT or 1298 CC genotypes were significantly more sensitized to OTX015 upon overexpression of wild-type MTHFR than their homozygous wild-type 677 CC and 1298 AA counterparts (FIG. 1G). We then reasoned that supplementation of cells with the end-product metabolite synthesized by MTHFR, 5-CH3 THF, may induce similar effect on cell response to OTX015 as wild-type MTHFR overexpression. Despite the increased IMS-M2 and U937 cell line resistance to OTX015 in response to MTHFR depletion, we thus observed that treatment with exogenous 5-CH3 THF restored a cytotoxic effect of OTX015 similar to that observed in shCT-infected cells (FIG. 1H). Consistent with this observation, exogenous 5-CH3 THF substantially enhanced response to OTX015 of homozygous 677 TT or 1298 CC MTHFR KG1a clones compared to their wild-type MTHFR counterparts (FIG. 1I). In addition, exogenous 5-CH3 THF sensitized to OTX015 primary patient cells exhibiting an altered MTHFR genotype (FIG. 1J).

Disruption of Folate Cycle Activity Increases Intracellular Levels of the Methyltransferase Inhibitor S-adenosylhomocysteine, and Impairs H3K27 and H3K9 Methyltransferase Activities.

We next used metabolism profiling to characterize the effect of folate pathway alteration on global cell metabolism in U937 cells. Steady state levels of 113 metabolites highly enriched in pathways either directly coupled to the folate cycle such as pyrimidine metabolism as well as histidine, glycine/serine/threonine, and cysteine/methionine/taurine/glutathione metabolisms, or indirectly associated with folate metabolism including urea cycle-related pathways such as arginine/proline, alanine, aspartate, glutamate, and beta-alanine metabolisms, were significantly changed by folate starvation (data not shown). Interestingly, this revealed a significant increase in S-adenosylhomocysteine (SAH), an intermediate in homocysteine synthesis from all methylation reactions involving the methionine-derived metabolite, S-adenosylmethionine (SAM), as methyl donor. ShRNA-mediated MTHFR depletion induced a similar increase in SAH levels as that observed upon folate withdrawal in AML cell lines U937 and IMS-M2 (data not shown). Finally, SAH supplementation increased OTX015 IC₅₀ by a magnitude similar to that measured upon folate starvation in KG1a and U937 cells (data not shown). Of note, depleting methionine in media of IMS-M2 cells (by 300-fold in methionine low “L” condition) enhanced the cytoprotective effect of SAH against OTX015 to a similar extent as that induced by FA starvation. In U937 and KG1a cells, this caused even greater resistance to OTX015 than folate restriction alone. Together, these data suggest that increased intracellular SAH is critical in mediating OTX015 resistance in response to impaired folate cycle activity.

Previous studies have reported that SAH is a potent inhibitor of SAM-dependent methylation reactions (19). We thus hypothesized that increased SAH levels induced by folate cycle suppression may directly impact methylation levels on histone H3. Profiling of the methylation status on H3 histone marks revealed that FA starvation significantly decreased dimethylation (me2) and trimethylation (me3) of H3K9 and H3K27, respectively (data not shown). Furthermore, MTHFR suppression reduced H3K27me3 and H3K9me2 both in human and murine AML cells (data not shown). This decreased methylation status resulted from a significant drop in the activity of H3K27 and H3K9 methyltransferase upon folate starvation or MTHFR depletion (data not shown).

To identify precisely the class of H3K27 and H3K9 methyltransferases whose decreased activity affect sensitivity to BET inhibitors, we deployed a CRISPR/Cas9 screening strategy using a dedicated library targeting 325 epigenetic regulators including DNA writers and erasers such as histone methylases/demethylases. sgRNA library-transduced cells were treated with JQ1 or DMSO, sampled weekly for 2 weeks of treatment to determine the composition of the sgRNA pools by deep sequencing. We identified a core component of the H3K27-methyltransferase PRC2 complex, EED, and two other SET-domain-containing H3K9 methyltransferases, EIMT1 and SETDB1, as top rescuer hits whose knockout rescued cells from JQ1 (data not shown). Conversely, knockout of three H3K9 demethylase enzymes, KDMIA, JMJDIC, and KDM3B, enhanced cell sensitivity to JQ1. To confirm these results, two hairpins that reduced EED, EHMT1, and SETB1 protein levels were transduced in U937 cells and markedly alleviated OTX015 sensitivity, phenocopying the cytoprotective effect of MTHFR depletion previously observed in BET inhibitor-treated cells (data not shown).

Folate Cycle Disruption Activates a SPI1 Transcriptional Program in Response to BET Inhibition.

Our data imply that folate cycle suppression reduces H3K27 and H3K9 methylation. This suggests that folate cycle disruption may induce transcriptomic and epigenetic rewiring of AML cells to activate a specific transcriptional program which mitigates the efficacy of BET inhibitors. We profiled the transcriptomes of two AML cell lines, IMS-M2 and U937, treated with OTX015 for 24 hours with or without folic acid, using RNA sequencing (RNAseq). Consistent with the fact that folate withdrawal decreases methylation of repressive H3K27me3 and H3K9me2 histone marks, we observed a global increase in gene expression, despite OTX015 treatment, which has been shown to preferentially repress genes marked by super-enhancers (412 and 706 significantly upregulated versus 160 and 520 significantly downregulated genes in IMS-M2 and U937 cells, respectively, data not shown). Accordingly, we identified 75 and 15 genes significantly 2-fold upregulated and downregulated between both folate-deprived cell lines, respectively, compared to cells in regular media with OTX015 (data not shown). An open-ended enrichment analysis was next conducted using i) a relaxed OTX015 signature generated from genes differentially expressed between DMSO and OTX015 conditions irrespective of folate status and ii) a relaxed gene signature representative of folate withdrawal irrespective of DMSO or OTX015 treatment (defined based on the cut-offs SNR permutation p value≤0.05, Benjamini-Hochberg false discovery rate (FDR)≤0.25 and absolute fold change for log 2 (FPKM) scores≥0.4) on the entire set of signatures from the c2, c3, and c6 (MsigDB) and ENCODE and CHEA collections (data not shown). Consistent with the reported effect of BRD4 inhibitors on MYC expression, we established that, irrespective of folate status, gene sets related to MYC transcriptional program were highly enriched (FDR<0.25) in genes whose expression is suppressed by OTX015 in the cell lines tested (data not shown). Conversely, many gene sets related to SPI1 transcriptional program and interferon signaling and regulatory factors (IRF), a well-reported SPI1-connected pathway (20, 21), were drastically enriched in genes whose expression is enhanced by folate withdrawal specifically with OTX015 treatment (data not shown). To investigate whether OTX015-induced activation of SPI1-related transcriptional programs reflects epigenetic changes induced by folate starvation, we profiled genome-wide distribution of H3K27me3 and H3K9me2 by chromatin immunoprecipitation (ChIP) sequencing of OTX015-treated IMS-M2 and U937 cells with regular or folate-restricted media. This confirmed that H3K27me3 binding signal in a 10 Kb-region flanking gene transcriptional starting sites (TSS) was decreased with folic acid starvation (data not shown). A total of 3868 and 4214 genes were annotated within the 50 kb-flanking H3K27me3 or H3K9me2-marked regions exhibiting a decreased methylation level with folate withdrawal in OTX015-treated IMS-M2 and U937 cells, respectively (data not shown). By overlapping these gene sets with genes identified by RNAseq as upregulated with folate starvation (530 and 1140 genes in IMS-M2 and U937 cells, respectively), we narrowed down the list of genes whose upregulation was directly associated with the H3K27me3 and H3K9me2 demethylation to 93 and 202 genes in IMS-M2 and U937 cells, respectively. These two gene lists were then queried by overlapping analysis with two ENCODE and CHEA datasets generated from curated target genes of transcription factors defined based on published ChIP-chip, ChIP-seq, and other transcription factor binding site profiling studies (data not shown). Two gene signatures related to SPI1 target genes from ENCODE and CHEA scored among the top enriched pathways, confirming that SPI1 transcriptional program activation in response to OTX015 treatment and folate withdrawal results from H3K27 and H3K9 demethylation. Finally, using the top genes from SPI1 target-related gene sets which were upregulated upon folate withdrawal and OTX015 treatment, we designed a SPI1 consensus transcriptional target mini signature. We used qRT-PCR to assess alteration of these signature genes and confirmed induction of these SPI1 target genes in response to MTHFR depletion and OTX015 treatment (data not shown).

To explore functionally whether SPI1 transcriptional program activation induced by folate cycle disruption promotes resistance to BRD4 inhibition, we knocked down SPI1 in IMS-M2 and U937 cells before folate withdrawal or MTHFR suppression (FIGS. 5F and 5G). SPI1 depletion significantly reduced OTX015 resistance in AML cells whose folate cycle activity was disturbed or MTHFR expression was suppressed (data not shown). Similarly, Spi1 knockdown abrogated resistance to OTX015 of MLL-AF9-transformed Mthfr knockout primary murine cells (data not shown). Together, these data suggest that folate cycle disruption promotes H3K27 and H3K9 demethylation, which activates a SPI1 transcriptional program capable of promoting resistance to BRD4 inhibition.

Discussion:

Here we revealed folate-connected metabolic gene signatures whose activation was poorly correlated with an active MYC transcriptional program in AML. This core metabolic network is composed of the histidine, serine, threonine and glycine pathways, which serve as inputs to the folate cycle (22), the beta-alanine/histidine synthesis pathway (23), and the phenylalanine- and DHRF-dependent tetrahydrobiopterin regeneration pathway (24). We also identified the tryptophan pathway as part of this core metabolic unit, likely because this pathway produces vitamin B6, an essential co-factor for synthesis of 5-CH3 THF from serine and THF (25).

Folate metabolism supports manifold transformations which depend on the oxidative state of THF, with 5,10-CH2-THF, 5-CH3-THF, and 10-CHO-THF each supporting distinct biosynthetic functions. 5,10-CH2-THF is used in three ways: 1) by serine hydroxymethyltransferase (SHMT1) or thymidylate synthase (TYMS) to synthesize serine or sustain de novo thymidylate synthesis, respectively; 2) by MTHFR for reduction to 5-CH3-THF and entrance into the folate cycle, which stimulates the methylation cycle; or, 3) by oxidation into 10-CHO-THF to sustain the THF salvage pathway which promotes purine synthesis and maintains mitochondrial redox homeostasis (22). Although a subset of folate- and MTHFR-related gene sets were poorly correlated with MYC signatures, we demonstrate that gene sets related to the THF salvage pathway and purine synthesis were strongly associated with active MYC gene signatures. This is consistent with previous studies showing that MYC promotes expression of SHMT2, MTHFD2, and MTHFD1L mitochondrial enzymes which contribute to purine production through the THF salvage pathway (26, 27). Despite the fact that both the folate cycle and tetrahydrofolate salvage pathways are interconnected within folate metabolism, our data hereby suggest that these pathways can be uncoupled on the basis of their connectivity to MYC transcriptional programs.

Consistent with this observation, we demonstrate functionally that folate starvation promotes resistance to MYC targeting by either BRD4 or CDK7 inhibitors, or BRD4-directed shRNAs. Previous studies suggest that folate cycle, de novo thymidylate synthesis, and THF salvage pathways compete for a limiting pool of 5,10-CH2-THF upon folate starvation. For instance, thymidylate synthesis is preserved in folate deficiency at the expense of the methylation cycle, which relies on activity of 5,10-CH2-THF-dependent MTHFR from the folate cycle. This favorable balance toward sustained thymidylate synthesis results from the activity of a critical cytosolic 10-CHO-THF-producer, MTHFD1, which translocates to the nucleus upon folate starvation to maintain de novo thymidylate biosynthesis (28, 9). Moreover, nuclear MTHFD1 also regulates transcription by direct binding to BRD4-occupied chromatin (30). These observations suggest that all folate-related metabolic pathways are not affected to the same extent by folate restriction. Although some pathways (10-CHO-THF-dependent reactions) persist without folate due to the plasticity of enzymes such as MTHFD1, the folate cycle and its associated methylation cycle are more severely impaired and thereby influence cell response to BET inhibitors.

We pinpoint MTHFR as the most critical folate-related mediator of resistance to BRD4 inhibitors and demonstrate that the presence of either of two MTHFR polymorphisms (677C>T; 1298A>C) in a homozygous state predicts sensitivity to BET inhibition. We observe that folate cycle disturbance in AML cells results in intracellular accumulation of SAH, the downstream effector of MTHFR knockdown triggering BET inhibitor resistance. Because the conversion of SAH to homocysteine is reversible but SAH-preferential, our results are consistent with the fact that hyperhomocysteinemia is accompanied by an elevation of SAH in Mthfr knockout animals (18, 31). Given that SAH is a potent inhibitor of SAM-dependent methylation, folate cycle impairment suppresses H3K27 and H3K9 methyltransferases and target methylation across the whole genome of AML cells. We validated that a SAM-dependent H3K27-methyltransferase PRC2 complex member, EED, scored among three downstream mediators of resistance to BET inhibitors, thereby recapitulating that suppression of the PRC2 complex promotes BET inhibitor resistance in AML (32).

Decreased H3K27 and H3K9 methylation upon folate cycle alteration combined with BET inhibition activates SPI1 and IRF/Interferon signaling transcriptional programs. These programs are tightly linked through physical interactions between SPI1 and either IRF4 or IRF8 in hematopoietic stem and progenitor cells (33, 34). We establish that SPI1 program activation is the main downstream effector of resistance to BET inhibitors with folate or MTHFR deficiency. Indeed, inhibition of demethylase KDM1A increases resistance to BRD4 inhibition via an IRF8/SPI1-mediated epigenetic rewiring (35). This supports the idea that the folate cycle dynamically influences epigenetic AML cell resistance by favoring an adaptive transcriptional plasticity relying on widespread, SPI1-dependent transcriptional changes. The latter acts as a compensatory mechanism to sustain cell survival despite BRD4 inhibition, a canonical epigenetic regulator of enhancer regions.

Two clinical trials are currently evaluating the clinical efficacy of combining BET inhibitors and Azacidine, the standard-of-care treatment of elderly AML patients (NCT02303782 and NCT02543879), thereby raising clinical interest in identifying relevant biomarkers of response to BET inhibitors. Beside folate and MTHFR deficiencies, deficits in folate cycle enzyme cofactors like cobalamin (vitamin B12) or pyridoxal phosphate (from vitamin B6) also affect methylation and cause hyperhomocysteinemia (36, 37). Up to 38% of older adults exhibit a food-cobalamin malabsorption syndrome characterized by B12 deficiency while the incidence of B6 deficiency in European institutionalized elderly people reaches up to 75%. Thus, our study likely underestimates the prevalence of folate cycle deficiency-induced BET inhibitor resistance among AML patients, who are mainly elderly. Additional work is required to evaluate to what extent the effect of folate/methylation cycle disruption upon B12, B6 deficiencies or loss-of-function mutations in enzymes controlling these metabolic pathways may, such as MTHFR insufficiency, promote resistance to MYC-targeting therapies.

Our study supports that MTHFR and folate cycle deficiencies remodel the epigenetic landscape to shape transcriptional plasticity which represents a cornerstone of resistance to epigenetic therapies. Their status should be carefully assessed by measurements of total plasma homocysteine or CH3-THF levels when enrolling patients with hematological diseases in clinical trials of BET inhibitors. Dietary supplementation with CH3-THF may thereby represent a relatively low-risk intervention that might allow for a greater clinical benefit to curtail transcriptional adaptation of malignant cells to MYC-targeting therapies.

### REFERENCES

Throughout this application, various references describe the state of the art to which this invention pertains. The disclosures of these references are hereby incorporated by reference into the present disclosure.
1. Hanahan D, Weinberg R A. Hallmarks of cancer: the next generation. Cell. 2011; 144:646-74.
2. Hsu P P, Sabatini D M. Cancer cell metabolism: Warburg and beyond. Cell. 2008; 134:703-7.
3. Lunt S Y, Vander Heiden M G. Aerobic glycolysis: meeting the metabolic requirements of cell proliferation. Annual review of cell and developmental biology. 2011; 27:441-64.
4. Pavlova N N, Thompson C B. The Emerging Hallmarks of Cancer Metabolism. Cell metabolism. 2016; 23:27-47.
5. Luengo A, Gui D Y, Vander Heiden M G. Targeting Metabolism for Cancer Therapy. Cell chemical biology. 2017; 24:1161-80.
6. Kanarek N, Keys H R, Cantor J R, Lewis C A, Chan S H, Kunchok T, et al. Histidine catabolism is a major determinant of methotrexate sensitivity. Nature. 2018; 559:632-6.
7. Stine Z E, Walton Z E, Altman B J, Hsieh A L, Dang C V. MYC, Metabolism, and Cancer. Cancer discovery. 2015; 5:1024-39.
8. Zuber J, Shi J, Wang E, Rappaport A R, Herrmann H, Sison E A, et al. RNAi screen identifies Brd4 as a therapeutic target in acute myeloid leukaemia. Nature. 2011; 478:524-8.
9. Chen H, Liu H, Qing G. Targeting oncogenic Myc as a strategy for cancer treatment. Signal transduction and targeted therapy. 2018; 3:5.
10. Puissant A, Frumm S M, Alexe G, Bassil C F, Qi J, Chanthery Y H, et al. Targeting MYCN in neuroblastoma by BET bromodomain inhibition. Cancer discovery. 2013; 3:308-23.
11. Delmore J E, Issa G C, Lemieux M E, Rahl P B, Shi J, Jacobs H M, et al. BET bromodomain inhibition as a therapeutic strategy to target c-Myc. Cell. 2011; 146:904-17.
12. Filippakopoulos P, Qi J, Picaud S, Shen Y, Smith W B, Fedorov O, et al. Selective inhibition of BET bromodomains. Nature. 2010; 468:1067-73.
13. Boi M, Gaudio E, Bonetti P, Kwee I, Bernasconi E, Tarantelli C, et al. The BET Bromodomain Inhibitor OTX015 Affects Pathogenetic Pathways in Preclinical B-cell Tumor Models and Synergizes with Targeted Drugs. Clinical cancer research: an official journal of the American Association for Cancer Research. 2015; 21:1628-38.
14. Devlin A M, Arning E, Bottiglieri T, Faraci F M, Rozen R, Lentz S R. Effect of Mthfr genotype on diet-induced hyperhomocysteinemia and vascular function in mice. Blood. 2004; 103:2624-9.
15. Mikael L G, Pancer J, Jiang X, Wu Q, Caudill M, Rozen R. Low dietary folate and methylenetetrahydrofolate reductase deficiency may lead to pregnancy complications through modulation of ApoAI and IFN-gamma in spleen and placenta, and through reduction of methylation potential. Molecular nutrition & food research. 2013; 57:661-70.
16. Ranganathan P, McLeod H L. Methotrexate pharmacogenetics: the first step toward individualized therapy in rheumatoid arthritis. Arthritis and rheumatism. 2006; 54:1366-77.
17. Weisberg I, Tran P, Christensen B, Sibani S, Rozen R. A second genetic polymorphism in methylenetetrahydrofolate reductase (MTHFR) associated with decreased enzyme activity. Molecular genetics and metabolism. 1998; 64:169-72.
18. Chen Z, Karaplis A C, Ackerman S L, Pogribny I P, Melnyk S, Lussier-Cacan S, et al. Mice deficient in methylenetetrahydrofolate reductase exhibit hyperhomocysteinemia and decreased methylation capacity, with neuropathology and aortic lipid deposition. Human molecular genetics. 2001; 10:433-43.
19. Chen N C, Yang F, Capecci L M, Gu Z, Schafer A L, Durante W, et al. Regulation of homocysteine metabolism and methylation in human and mouse tissues. FASEB journal: official publication of the Federation of American Societies for Experimental Biology. 2010; 24:2804-17.
20. Kubosaki A, Lindgren G, Tagami M, Simon C, Tomaru Y, Miura H, et al. The combination of gene perturbation assay and ChIP-chip reveals functional direct target genes for IRF8 in THP-1 cells. Molecular immunology. 2010; 47:2295-302.
21. Will B, Vogler T O, Narayanagari S, Bartholdy B, Todorova T I, da Silva Ferreira M, et al. Minimal PU.1 reduction induces a preleukemic state and promotes development of acute myeloid leukemia. Nature medicine. 2015; 21:1172-81.
22. Ducker G S, Rabinowitz J D. One-Carbon Metabolism in Health and Disease. Cell metabolism. 2017; 25:27-42.
23. Boldyrev A A, Aldini G, Derave W. Physiology and pathophysiology of carnosine. Physiological reviews. 2013; 93:1803-45.
24. Shi W, Meininger C J, Haynes T E, Hatakeyama K, Wu G. Regulation of tetrahydrobiopterin synthesis and bioavailability in endothelial cells. Cell biochemistry and biophysics. 2004; 41:415-34.
25. Badawy A A. Kynurenine Pathway of Tryptophan Metabolism: Regulatory and Functional Aspects. International journal of tryptophan research: IJTR. 2017; 10:1178646917691938.
26. Nikiforov M A, Chandriani S, O'Connell B, Petrenko O, Kotenko I, Beavis A, et al. A functional screen for Myc-responsive genes reveals serine hydroxymethyltransferase, a major source of the one-carbon unit for cell metabolism. Molecular and cellular biology. 2002; 22:5793-800.
27. Ye J, Fan J, Venneti S, Wan Y W, Pawel B R, Zhang J, et al. Serine catabolism regulates mitochondrial redox control during hypoxia. Cancer discovery. 2014; 4:1406-17.
28. Field M S, Kamynina E, Agunloye O C, Liebenthal R P, Lamarre S G, Brosnan M E, et al. Nuclear enrichment of folate cofactors and methylenetetrahydrofolate dehydrogenase 1 (MTHFD1) protect de novo thymidylate biosynthesis during folate deficiency. The Journal of biological chemistry. 2014; 289:29642-50.
29. Green J M, MacKenzie R E, Matthews R G. Substrate flux through methylenetetrahydrofolate dehydrogenase: predicted effects of the concentration of methylenetetrahydrofolate on its partitioning into pathways leading to nucleotide biosynthesis or methionine regeneration. Biochemistry. 1988; 27:8014-22.
30. Sdelci S, Rendeiro A F, Rathert P, You W, Lin J G, Ringler A, et al. MTHFD1 interaction with BRD4 links folate metabolism to transcriptional regulation. Nature genetics. 2019; 51:990-8.
31. Castro R, Rivera I, Struys E A, Jansen E E, Ravasco P, Camilo M E, et al. Increased homocysteine and S-adenosylhomocysteine concentrations and DNA hypomethylation in vascular disease. Clinical chemistry. 2003; 49:1292-6.
32. Rathert P, Roth M, Neumann T, Muerdter F, Roe J S, Muhar M, et al. Transcriptional plasticity promotes primary and acquired resistance to BET inhibition. Nature. 2015; 525:543-7.
33. Pang S H, Minnich M, Gangatirkar P, Zheng Z, Ebert A, Song G, et al. PU.1 cooperates with IRF4 and IRF8 to suppress pre-B-cell leukemia. Leukemia. 2016; 30:1375-87.
34. Pham T H, Minderjahn J, Schmidl C, Hoffmeister H, Schmidhofer S, Chen W, et al. Mechanisms of in vivo binding site selection of the hematopoietic master transcription factor PU.1. Nucleic acids research. 2013; 41:6391-402.
35. Bell C C, Fennell K A, Chan Y C, Rambow F, Yeung M M, Vassiliadis D, et al. Targeting enhancer switching overcomes non-genetic drug resistance in acute myeloid leukaemia. Nature communications. 2019; 10:2723.
36. Kjeldby I K, Fosnes G S, Ligaarden S C, Farup P G. Vitamin B6 deficiency and diseases in elderly people—a study in nursing homes. BMC geriatrics. 2013; 13:13.
37. Stover P J. Vitamin B12 and older adults. Current opinion in clinical nutrition and metabolic care. 2010; 13:24-7.
38. Fenouille N, Bassil C F, Ben-Sahra I, Benajiba L, Alexe G, Ramos A, et al. The creatine kinase pathway is a metabolic vulnerability in EVI1-positive acute myeloid leukemia. Nature medicine. 2017; 23:301-13.
39. McLean C Y, Bristor D, Hiller M, Clarke S L, Schaar B T, Lowe C B, et al. GREAT improves functional interpretation of cis-regulatory regions. Nature biotechnology. 2010; 28:495-501.
40. Cancer Genome Atlas Research N, Ley T J, Miller C, Ding L, Raphael B J, Mungall A J, et al. Genomic and epigenomic landscapes of adult de novo acute myeloid leukemia. The New England journal of medicine. 2013; 368:2059-74.

1. A method of determining whether a patient suffering from acute myeloid leukemia will achieve a response to an MYC-targeting therapy comprising determining in a nucleic acid sample obtained from the subject the presence or absence of at least one genetic variant in the MTHFR gene wherein the presence of said genetic variant indicates that the patient will not achieve a response to the MYC-targeting therapy whereas the absence of said genetic variant indicates that the patient will achieve a response to the MYC targeting therapy.

2. The method of claim 1 wherein the MYC-targeting therapy is selected from the group consisting of inhibitors of MYC transcription with direct G-quadruplex stabilizers, antisense oligonucleotides that induce MYC mRNA degradation, aberrant splicing of MYC pre-mRNA or translation block, and short-interfering RNAs.

3. The method of claim 1 wherein the MYC targeting therapy is selected from the group consisting of bromodomain inhibitors, CDK7 inhibitors, and CDK9 inhibitors.

4. The method of claim 3 wherein the bromodomain inhibitor is a BET inhibitor.

5. The method of claim 4 wherein the BET inhibitor is selected from the group consisting of RVX-208, PFI-1, OTX015, BzT-7, GSK525762A, JQ1, I-BET-762, and LY294002.

6. The method of claim 1 wherein the presence or absence of the c.677C>T or c.1298A>C is determined, and wherein the presence of 677 CC or 1298 AA genotype indicate that the patient will not achieve a response to the MYC-targeting therapy.

7. A method of treating acute myeloid leukemia in a patient in need thereof comprising
i) determining in a nucleic acid sample obtained from the subject the presence or absence of at least one genetic variant in the MTHFR gene and
ii) administering to the patient a MYC-targeting therapy when the at least one genetic variant is not detected or administering to the patient a MYC-targeting therapy in combination with a therapeutically effective amount of 5-methyltetrahydrofolate when the at least one genetic variant is detected.

8. The method of claim 7 wherein the 5-methyltetrahydrofolate is administered by dietary supplementation.

9. The method of claim 7 wherein the MYC-targeting therapy is selected from the group consisting of inhibitors of MYC transcription with direct G-quadruplex stabilizers, antisense oligonucleotides that induce MYC mRNA degradation, aberrant splicing of MYC pre-mRNA or translation block, and short-interfering RNAs.

10. The method of claim 7 wherein the MYC targeting therapy is selected from the group consisting of bromodomain inhibitors, CDK7 inhibitors, and CDK9 inhibitors.

11. The method of claim 10 wherein the bromodomain inhibitor is a BET inhibitor.

12. The method of claim 11 wherein the BET inhibitor is selected from the group consisting of RVX-208, PFI-1, OTX015, BzT-7, GSK525762A, JQ1, I-BET-762, and LY294002.

13. The method of claim 7 wherein the presence or absence of the c.677C>T or c.1298A>C is determined, and wherein the presence of 677 CC or 1298 AA genotype indicate that the patient will not achieve a response to the MYC-targeting therapy.
